(19) United States
(12) Patent Application Publication
(10) Pub. No.: US 2017/0105635 A1
(43) Pub. Date: Apr. 20, 2017

(54) INTRACARDIAC MEDICAL DEVICE WITH PRESSURE SENSING

(71) Applicant: Medtronic, Inc., Minneapolis, MN (US)

(72) Inventors: Yong K. Cho, Excelsior, MN (US); Michael F. Hess, Minneapolis, MN (US); Leonardo Rapallini, Edina, MN (US); Todd J. Sheldon, North Oaks, MN (US); Brian D. Urke, Medina, MN (US)

(21) Appl. No.: 14/934,466

(22) Filed: Nov. 6, 2015

Related U.S. Application Data
(60) Provisional application No. 62/242,554, filed on Oct. 16, 2015.

(51) Int. Cl. A61B 5/0215; A61N 1/362; A61B 5/042; A61B 5/021; A61B 5/00

(52) U.S. Cl. CPC A61B 5/0215; A61B 5/02141; A61B 5/686; A61B 5/6869; A61B 5/0422; A61N 1/3621

(57) ABSTRACT

An implantable medical device includes a housing having a proximal end and a distal end, a control module enclosed by the housing, and a pressure sensor electrically coupled to the control module. A fixation member is coupled to the housing distal end for anchoring the housing distal end at a fixation site within a cardiovascular system of a patient, and the pressure sensor is spaced apart proximally from the fixation member.

### CROSS-REFERENCE TO RELATED APPLICATIONS

This application claims the benefit of U.S. Provisional Application No. 62/242,554, filed on Oct. 16, 2015. The disclosure of the above application is incorporated herein by reference in its entirety.

### TECHNICAL FIELD

The disclosure relates to an implantable intracardiac medical device for monitoring pressure of circulating blood.

### BACKGROUND

A variety of implantable medical devices (IMDs) for delivering a therapy and/or monitoring a physiological condition have been clinically implanted or proposed for clinical implantation in patients. Some IMDs employ sensors for monitoring physiological signals such as pressure, temperature, pH, oxygen saturation or other signals. IMDs may employ electrodes for monitoring electrical signals such as the electrocardiogram (ECG). IMDs may deliver electrical stimulation or pharmacologic therapy to the heart, muscle, nerve, brain, stomach or other organs or tissue, as examples. IMDs, such as cardiac pacemakers or implantable cardioverter defibrillators, for example, provide therapeutic electrical stimulation to the heart via electrodes carried by one or more implantable leads. The IMD is typically implanted in a subcutaneous pocket from which medical electrical leads coupled to the IMD extend, e.g., transvenously, to locations within or along the heart.

### SUMMARY

In general, the disclosure is directed to an IMD for monitoring pressure of circulating blood. An IMD according to the present disclosure includes a housing having a distal fixation member and a pressure sensor that is located proximally from the distal fixation member for monitoring pressure of circulating blood within the cardiovascular system at a location spaced apart from the distal fixation member.

In one example, the disclosure provides an IMD including a housing having a proximal end and a distal end, a control module enclosed by the housing, a pressure sensor electrically coupled to the control module, and a fixation member coupled to the housing distal end for anchoring the housing distal end at an implant site within a cardiovascular system of a patient. The pressure sensor is spaced apart proximally from the fixation member.

In another example, the disclosure provides a system including an IMD and a delivery tool. The IMD includes a housing having a proximal end and a distal end, a control module enclosed by the housing, a pressure sensor electrically coupled to the control module, a fixation member coupled to the housing distal end for anchoring the housing distal end at an implant site within a cardiovascular system of a patient. The pressure sensor is spaced apart proximally from the fixation member. The delivery tool includes a cavity for receiving the housing and for advancing the housing distal end to the implant site.

In another example, the disclosure provides an IMD including a housing having a proximal end and a distal end, a control module enclosed by the housing, an electrical extension having a distal end extending from the housing proximal end, a free proximal end, and an elongate body extending from the distal end to the free proximal end. A pressure sensor is carried by the electrical extension elongate body and electrically coupled to the control module via the electrical extension. The IMD includes a fixation member for anchoring the implantable medical device at an implant site within a cardiovascular system of a patient. The fixation member is coupled to the housing distal end. The electrical extension carrying the pressure sensor extends to a pressure monitoring site within a volume of circulating blood without fixation of the IMD to tissue at the pressure monitoring site. The pressure sensor is spaced apart from the housing when the housing is fixed at the implant site by the fixation member.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below

### BRIEF DESCRIPTION OF DRAWINGS

FIG. 1 is a conceptual diagram illustrating an implantable medical device (IMD) that may be used to monitor pressure of circulating blood in a patient.

FIG. 2A is a conceptual diagram of the IMD of FIG. 1 according to one example.

FIG. 2B is a schematic diagram of the IMD of FIG. 2A loaded into a delivery tool according to one example.

FIG. 2C is a conceptual diagram of the IMD of FIG. 1 according to another example.

FIG. 3A is a conceptual diagram of an IMD including a sensor extension carrying a pressure sensor and floatation member according to another example.

FIG. 3B is a floatation member that may be included on a sensor extension according to one example.

FIG. 3C is an end view of a floatation member incorporating a pressure sensor according to another example.

FIG. 4A is a conceptual diagram of an IMD including a proximal sensor extension and a floatation member.

FIG. 4B is a proximal end view of the floatation member of FIG. 4A.

FIG. 4C is a partial view of the sensor extension of FIG. 4A according to another example.

FIGS. 5-7 are conceptual diagrams of a delivery tool and an IMD as the IMD is deployed to a fixation site and a pressure monitoring site spaced apart from the fixation site.

FIGS. 8A and 8B are conceptual diagrams of alternative examples of an IMD having a pressure sensor incorporated within the IMD housing.

FIG. 9 is a block diagram of an IMD configured to monitor a pressure signal according to one example.

FIG. 10 is a flow chart of one method for deploying an IMD for monitoring a pressure signal.

### DETAILED DESCRIPTION

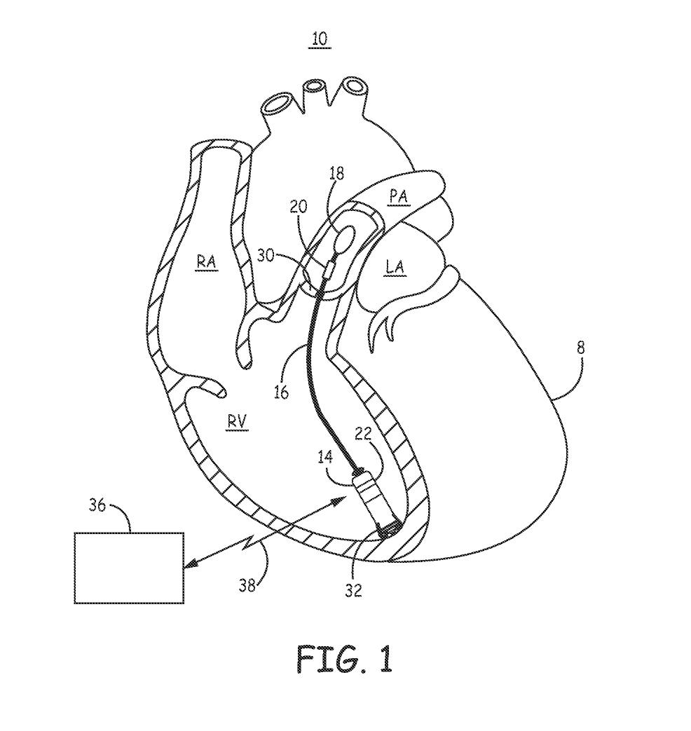

FIG. 1 is a conceptual diagram illustrating an implantable medical device (IMD) 10 that may be used to monitor pressure of circulating blood within a patient's cardiovascular system. IMD 10 is an elongated device that includes a housing 14 and a proximal sensor extension 16 extending from housing 14 and carrying a pressure sensor 18. The housing 14 encloses electronic circuitry, such as a control module for controlling pressure sensor 18 and determining a pressure parameter.

IMD 10 is shown deployed in the right ventricle (RV) of a patient's heart 8 with sensor extension 16 extending from the RV, across pulmonary valve 30, into the pulmonary artery (PA) so that in the example shown IMD 10 is positioned for monitoring pulmonary artery pressure (PAP) in a patient. IMD 10 may be delivered transvenously by a catheter advanced into the right atrium (RA) and into the RV. IMD 10 may include a distal fixation member 32, shown as multiple curved tines, for fixing housing 14 at a desired location in the RV, e.g., along the RV apex, upon release from the delivery catheter.

Upon release from a delivery catheter, the proximal sensor extension 16 drifts downstream in circulating blood along the right ventricular outflow tract (RVOT) into the PA. Pressure sensor 18 drifts downstream along the RVOT with blood ejected from the RV into the PA thereby extending sensor extension 16 from the RV into the PA. Sensor extension 16 is an electrical extension that couples pressure sensor 18 to electronic circuitry included in IMD housing 14.

IMD 10 may be configured to sense cardiac electrical signals in addition to sensing a signal from pressure sensor 18. Accordingly, IMD 10 may include a pair of sensing electrodes 20 and 22 carried along housing 14 and/or sensor extension 16. In the example shown, one sensing electrode 20 is carried by sensor extension 16 and one sensing electrode 22 is carried by housing 14. In other examples, both electrodes 20 and 22 may extend along housing 14 or both electrodes 20 and 22 may be carried by sensor extension 16. IMD 10 may be configured to sense cardiac electrical signals (e.g., including R-waves and P-waves) using the electrodes 20 and 22 for use in monitoring the patient's heart along with PAP. Electrodes 20 and 22 may be electrically coupled to an electrical sensing module enclosed by IMD housing 14 as further described below.

IMD 10 may be configured as a therapy delivery device in some examples. For example, IMD 10 may be configured to deliver cardiac electrical stimulation pulses, such as bradycardia pacing pulses, cardiac resynchronization pacing pulses, or anti-tachyarrhythmia pacing therapy. In such examples, electrodes 20 and 22 may be electrically coupled to a pulse generator enclosed by housing 14 for delivering electrical stimulation pulses.

IMD 10 may be configured for bidirectional wireless telemetry with an external device 36. External device 36 may be a programmer, home monitor, or handheld device. External device 36 may be used to transfer data to and receive data from IMD 10 via a wireless radio frequency (RF) communication link 38 established using BLUETOOTH®, Wi-Fi, Medical Implant Communication Service (MICS) or other RF bandwidth. In some examples, external device 36 may include a programming head that is placed proximate IMD 10 to establish and maintain a communication link, and in other examples external device 36 and IMD 10 may be configured to communicate using a distance telemetry algorithm and circuitry that does not require the use of a programming head and does not require user intervention to maintain a communication link.

Aspects of external device 36 may generally correspond to the external programming/monitoring unit disclosed in U.S. Pat. No. 5,507,782 (Kieval, et al.), hereby incorporated herein by reference in its entirety. External device 36 is often referred to as a “programmer” because it is typically used by a physician, technician, nurse, clinician or other qualified user for programming operating parameters in IMD 10 as well as retrieving information about the patient or device, including retrieving a pressure signal or pressure parameter derived from a pressure signal acquired using sensor 18. External device 36 may be located in a clinic, hospital or other medical facility. External device 36 may alternatively be embodied as a home monitor or a handheld device that may be used in a medical facility, in the patient's home, or another location. Operating parameters, such as sensing and therapy delivery control parameters, may be programmed into IMD 10 using external device 36.

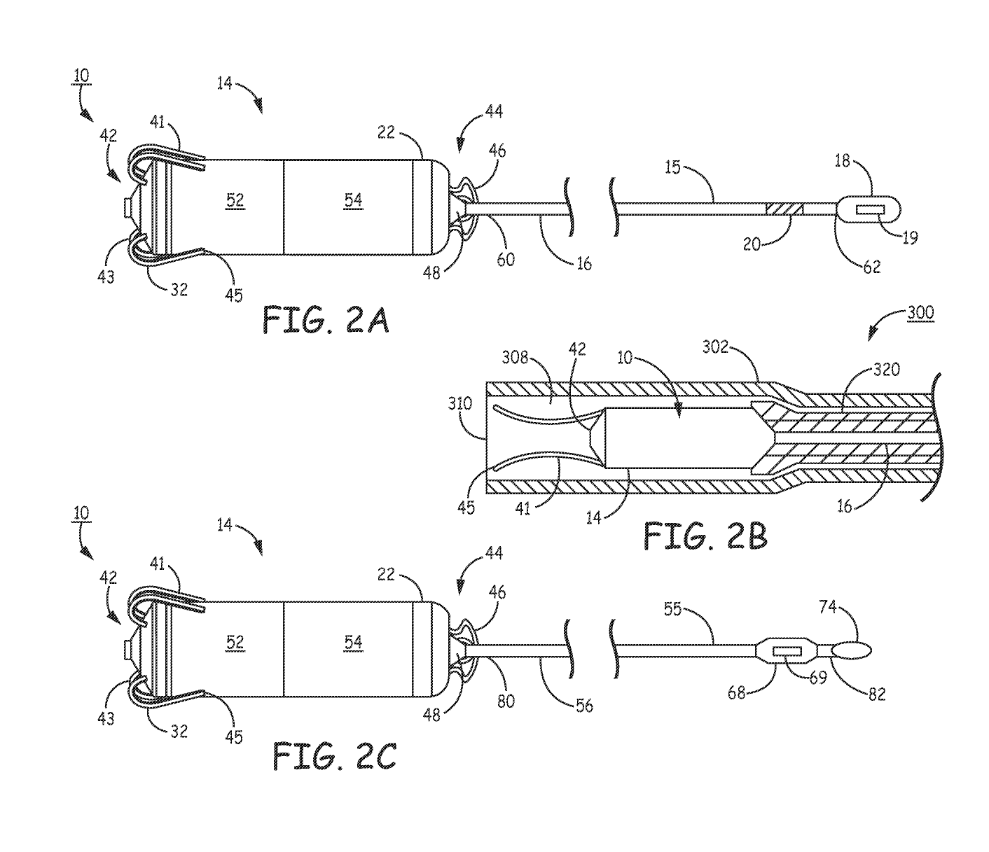

FIG. 2A is a conceptual diagram of IMD 10 according to one example. IMD 10 includes housing 14, having a housing distal end 42 and a housing proximal end 44, and a proximal sensor extension 16 attached to housing proximal end 44 and extending in a generally proximal direction therefrom. Distal end 42 is referred to as “distal” in that it is expected to be the leading end as IMD 10 is advanced to an implant site using a delivery tool, such as a catheter, and placed against a target implant site. Sensor extension 16 may be non-removably attached to housing 14 via a transition member 48. In other examples, sensor extension 16 may be removable and attachable by a user.

IMD housing 14 includes a control electronics subassembly 54, which encloses electronic circuitry for controlling IMD functions including sensing pressure signals from pressure sensor 18. Housing 14 further includes a battery subassembly 52, which provides power to the control electronics subassembly 54. Battery subassembly 52 may include features of the batteries disclosed in commonly-assigned U.S. Pat. No. 8,433,409 (Johnson, et al.) and U.S. Pat. No. 8,541,131 (Lund, et al.), both of which are hereby incorporated by reference herein in their entirety.

A fixation member 32 is coupled to housing distal end 42. Fixation member 32 may include multiple fixation tines 41 projecting from distal housing end 42 to stably maintain distal housing end 42 at an implant site by actively engaging with tissue at the implant site. The implant site may be within the patient's cardiovascular system, e.g., along a heart chamber such as the RV endocardium or along the RVOT, accessed via a transvenous catheter.

Fixation member tines 41 are shown spaced circumferentially along a periphery of the housing distal end 42, along battery subassembly 52 in this example. Each of fixation tines 41 may extend in a generally distal direction from a fixed tine end 43 that is fixedly coupled to housing distal end 42, then curve or bend laterally and proximally to a free, terminal tine end 45 that extends in a relatively proximal direction with respect to distal housing end 42.

Fixation member tines 41 are shown in a relaxed position in FIG. 2A and may be elastically deformed to an extended position during implantation of IMD 10 as shown schematically in FIG. 2B. FIG. 2B is a partial view of a delivery tool 300 with IMD 10 loaded within the delivery tool for deployment to an implant site. During implantation, IMD 10 may be placed in a delivery tool 300 such that fixation member tines 41 are held in a distally extended position. Delivery tool 300 may include an outer catheter 302 and an inner catheter 320 in some examples. Housing 14 may be retained within a cavity 308 defined by outer catheter 302. The inner catheter 320 may extend within the outer catheter 302 and define an inner lumen in which proximal sensor extension 16 may extend when IMD 10 is loaded in the delivery tool 300.

In the extended position as shown in FIG. 2B, the fixation member tines 41 are straightened in a distal direction from the relaxed curved position shown so that free tine ends 45 extend distally from housing distal end 42. The fixation member 32 may be an active fixation member having free tine ends 45 that pierce and advance through tissue at the implant site to maintain a stable position of housing 14. Upon ejection from the delivery tool 300 through open distal end 310, the free tine ends 45 first pierce tissue at the implant site then curve back proximally capturing tissue as the relaxed position of tines 41 is regained. In this way, fixation member 32 becomes fixedly engaged with tissue, anchoring housing distal end 42 to the implant site.

Fixation member 32 may be formed from a biocompatible polymer, e.g., polyurethane, silicone, polyethylene, or polyether ether ketone (PEEK). In some examples, fixation member 32 includes a shape memory material such as nitinol to retain a pre-formed bend or curve that is straightened when IMD 10 is placed in a delivery catheter or tool and restored after IMD 10 is released from the delivery catheter or tool. One or more tines 41 may include a radio-opaque marker that is visible under fluoroscopy or x-ray and facilitates delivery IMD 10 to a desired implant site and confirmation of fixation at a targeted site. Fixation member 32 extending from battery subassembly 52 may generally correspond to examples of a fixation member assembly disclosed in commonly-assigned U.S. patent application Ser. No. 14/518,261 (Eggen, et al.), and in commonly-assigned U.S. Publication No. 2012/0172892 (Grubac, et al.), both incorporated herein by reference in their entirety.

Sensor extension 16 includes a flexible extension body 15 extending from an extension distal end 60 to a free proximal end 62 extending away from housing 14. Extension distal end 60 may be fixedly coupled to housing proximal end 44. Pressure sensor 18 may be carried by sensor extension 16 at its free proximal end 62, e.g., terminating the proximal end of extension body 15. In other examples, pressure sensor 18 may be located distally to the free proximal end 62 of sensor extension 16.

Pressure sensor 18 may be provided with outer dimensions that are advanceable within a delivery tool used to deploy IMD 10 at an implant location. Pressure sensor 18 and flexible extension body 15 are not provided with a fixation member in some examples such that the free proximal end 62 that carries pressure sensor 18 floats in the patient's blood stream, away and downstream from an implant site of housing 14 when fixation member 32 is anchored within the patient's cardiovascular system. For example, when housing fixation member 32 is fixed within the RV, sensor extension 16 is pulled into the RVOT by circulating blood flowing out of the RV. The overall length of sensor extension 16 may be selected so that when fixation member 32 is anchored in the RV apex or another target implant site, sensor extension body 15 crosses the pulmonary valve such that pressure sensor 18 floats within the PA and produces a PAP signal.

When properly deployed at a targeted implant site, fixation member 32 has a fixation force that is greater than the opposing force against pressure sensor 18 caused by blood flow. For example, when fixation member 32 is deployed at a target site in the RV or the RVOT, the force of circulating blood acting against pressure sensor 18 that pulls sensor extension 16 into the RVOT and maintains the position of pressure sensor 18 in the PA is less than the fixation force of fixation member 32. Pressure sensor 18 may be maintained at a target pressure monitoring site without requiring a fixation member, active or passive, engaging pressure sensor 18 with tissue at the pressure monitoring site. A single fixation member 32 (which may include multiple tines 41) anchors the housing distal end of IMD 10 at the fixation site within the cardiovascular system of the patient. Pressure sensor 18 extends to the pressure monitoring site within a volume of circulating blood without fixation of the pressure sensor 18 to tissue at the pressure monitoring site, which is spaced apart from the housing 14 and from the fixation site.

Extension body 15 may be a flexible tube or multi-lumen body through which electrical conductors, e.g., cabled or multi-filar conductors, extend from pressure sensor 18 (and electrode 20 when included) to housing 14. Necessary electrical connections to circuitry included in control electronics subassembly 54 are provided via an electrical feedthrough (not shown in FIG. 2) crossing housing 14. Extension body 15 may be formed from silicone, polyurethane, polytetrafluorethylene (PTFE), or other biocompatible polymer material. Extension body 15 may be a highly flexible body that naturally aligns with flowing blood to extend “downstream” from housing 14 without resistance.

In some examples, extension body 15 has a variable stiffness that has the greatest stiffness along a distal portion nearest extension distal end 60 and the least or lowest stiffness nearest free proximal end 62. A variable stiffness may be achieved by forming extension body 15 with a material having higher stiffness near distal end 60 and a material having lower stiffness near proximal end 62, by adding a stiffening layer or coating near distal end 60, or by adding a stiffing member such as a rod, helical coil or other member extending along a portion of extension body 15 near extension distal end 60.

A stiff distal portion of extension body 15, e.g., adjacent distal end 60, may be self-supporting such that it assumes a pre-formed shape, which may be straight as shown in FIG. 2A or include a bend or curve. The stiffness of the self-supporting distal portion that makes it a self-supporting member may gradually decrease toward proximal end 62 such that proximal end 62 is highly flexible and compliant. The self-supporting distal portion adjacent extension distal end 60 promotes self-orienting of extension 16 in a desired direction away from housing 14 and minimizes flexion of and stresses on the extension body 15 in circulating blood while the proximal end 62 is relatively soft and flexible to avoid causing injury or trauma to a blood vessel wall, e.g., the inner wall of the PA. As indicated above, the self-supporting distal portion may include a pre-formed bend or curve that directs the more flexible proximal end toward the RVOT. Blood flowing past the proximal end 62 maintains pressure sensor 18 in a desired pressure-monitoring location without requiring fixation of sensor 18 or extension proximal end 62 in tissue of the inner wall of the blood vessel or heart.

Pressure sensor 18 may be implemented as a capacitive pressure transducer including a pressure-sensitive diaphragm 19 that is exposed to circulating blood. Pressure sensor 18 may be a microelectromechanical system (MEMS) sensor including a gap capacitor having a diaphragm electrode and a signal electrode producing an electrical signal correlated to surrounding pressure acting on diaphragm 19 and changing the gap between the diaphragm and signal electrodes. Aspects of a capacitive pressure transducer that may be included in pressure sensor 18 are generally disclosed in U.S. Pat. No. 8,424,388 (Mattes, et al.), incorporated herein by reference in its entirety. Pressure sensor 18 is not limited to being a capacitive pressure sensor and other types of transducers may be used to produce an electrical signal correlated to pressure exerted on sensor 18 by circulating blood.

Distal electrode 22, shown as a ring electrode, may be coupled to housing 14 to serve as a return electrode, that may be paired with proximal electrode 20 for sensing cardiac electrical signals. Proximal electrode 20, shown as a ring electrode carried by sensor extension 16, may be coupled to a sensing module within control electronics subassembly 54 via an electrical feedthrough. A cardiac EGM signal may be received by control electronics subassembly 54 for monitoring cardiac electrical activity in conjunction with pressure.

Electrodes 20 and 22 may be, without limitation, titanium, platinum, iridium or alloys thereof and may include a low polarizing coating, such as titanium nitride, iridium oxide, ruthenium oxide, platinum black among others. In alternative embodiments, IMD 10 may include two or more electrodes exposed along housing 14 and/or along sensor extension 16.

Housing 14 is formed from a biocompatible material, such as a stainless steel or titanium alloy. In some examples, the housing 14 may include an insulating coating. The entirety of the housing 14 may be insulated, but only electrode 22 uninsulated. Examples of insulating coatings include parylene, urethane, PEEK, or polyimide among others. In other examples, an insulating coating of housing 14 is not provided, but electrode 22 is electrically isolated from the remainder of housing 14.

IMD 10 may optionally include a delivery tool interface 46. Delivery tool interface 46 may be located at the proximal end 44 of IMD 10 and is configured to connect to a delivery device, such as a catheter, used to position IMD 10 at an implant location during an implantation procedure, for example within a heart chamber.

A reduced size of housing 14 of IMD 10 enables implantation of housing 14 wholly within a patient's heart such that housing 14 may be fixed in one heart chamber, e.g., the RV. The length of sensor extension 16 enables extension proximal end 62 to extend into the PA when housing distal end 42 is fixed at a target site. For example, with no limitation intended, housing 14 may have a length between housing distal end 42 and housing proximal end 44 in the range of and including approximately 1 to 5 cm. Sensor extension 16 may have a length between extension distal end 60 and extension proximal end 62 of approximately 3 to 15 cm so that pressure sensor 18 is “floated” near the pulmonary valve 30 (either before or after the pulmonary valve). In various examples, sensor extension 16 may be approximately 2 cm to 15 cm in length, depending on the overall length of housing 14 and distance between the targeted fixation site of housing 14 and the desired pressure monitoring site of sensor 18 among other considerations. The overall length of IMD 10 from housing distal end 42 to sensor extension proximal end 62 may be selected to enable implantation of housing 14 wholly in one heart chamber or blood vessel, e.g., the RV, with sensor extension 16, extending toward or within another heart chamber or blood vessel, e.g., within the PA.

FIG. 2C is a conceptual diagram of IMD 10 according to another example. In this example, IMD 10 includes a sensor extension 56 extending from the IMD housing proximal end 44 that includes a floatation member 74. Sensor extension 56 includes a sensor extension body 55 that extends from a sensor extension proximal end 80 to sensor extension distal end 82. A pressure sensor 68 is carried along the sensor extension body 55, intermediate the sensor extension proximal and distal ends 82 and 80, respectively. Pressure sensor 68 may be implemented as a capacitive pressure transducer including a diaphragm 69 for transferring surrounding pressure to internal pressure-sensitive capacitive elements.

Floatation member 74 is shown terminating sensor extension proximal end 82. In other examples, pressure sensor 68 may be located at the sensor extension proximal end 82 and floatation member 74 may be carried along extension body 55 distal to proximal end 82.

Floatation member 74 may be positively buoyant in blood or may be at least neutrally buoyant in blood. As used herein, the term “at least neutrally buoyant in blood” refers to a buoyancy of the floatation member 74 in blood that is neutral or positively buoyant and is not negatively buoyant in blood. In some examples, the buoyancy of floatation member 74 is selected to promote floatation of the proximal end of sensor extension 56 into the PA that does not create a buoyant force or pulling force due to pressure of circulating blood acting on floatation member 74 that is greater than the fixation force of distal fixation member 32. For example, distal fixation member 32 including multiple fixation times may have a fixation force that is greater than 0.5 Newtons per tine. Sensor extension 56 with floatation member 18 may have a pulling force opposing the fixation force of distal member 32 that is less than 0.5 Newtons when sensor extension 56 is subjected to flowing blood after implantation. For example, the pulling force of sensor extension with floatation member 18 when positioned in flowing blood may be approximately 0.01 N to 0.5 N.

Floatation member 74 may be a deformable or non-deformable hollow member, such as a silicone or polyurethane balloon that is filled with air or another lightweight material such as a polyurethane, polyethylene or silicone-based foam. In other examples, floatation member is a deformable or non-deformable solid member molded from a biocompatible material having a density lower than blood. The density of whole blood is approximately 1.06 g/cm³. Floatation member 74 may be molded from a biocompatible polymer, such as polyethylene or polypropylene, having a density less than 1.0 g/cm³ in some examples. Floatation member 74 may include an anti-thrombotic or other coating that reduces the adhesion of cells to floatation member 74 and promotes sliding of blood cells along the surface of floatation member 74. For example, floatation member 74 may have a highly smooth, hydrophilic or neutral surface coating to reduce encapsulation, which may include, with no limitation intended, a hydrogel, polytetrafluoroethylene or GORE-TEX® membrane material.

In some examples, all or a portion of floatation member 74 and other examples of floatation members shown and described herein is formed of a bioabsorbable material, e.g., polylactic acid (PLA), polyglycolic acid (PGA), PLA/PGA copolymers, or polycaprolactone (PCL). Floatation member 74 may be provided to hold sensor extension 56 in an extended position in flowing blood acutely after implantation, but over a period of time, e.g., weeks or months, tissue encapsulation of any portion of sensor extension 56 may maintain adequate stability of the position of pressure sensor 68 along the PA (or other desired pressure monitoring site). Floatation member 74 may be absorbed entirely or partially.

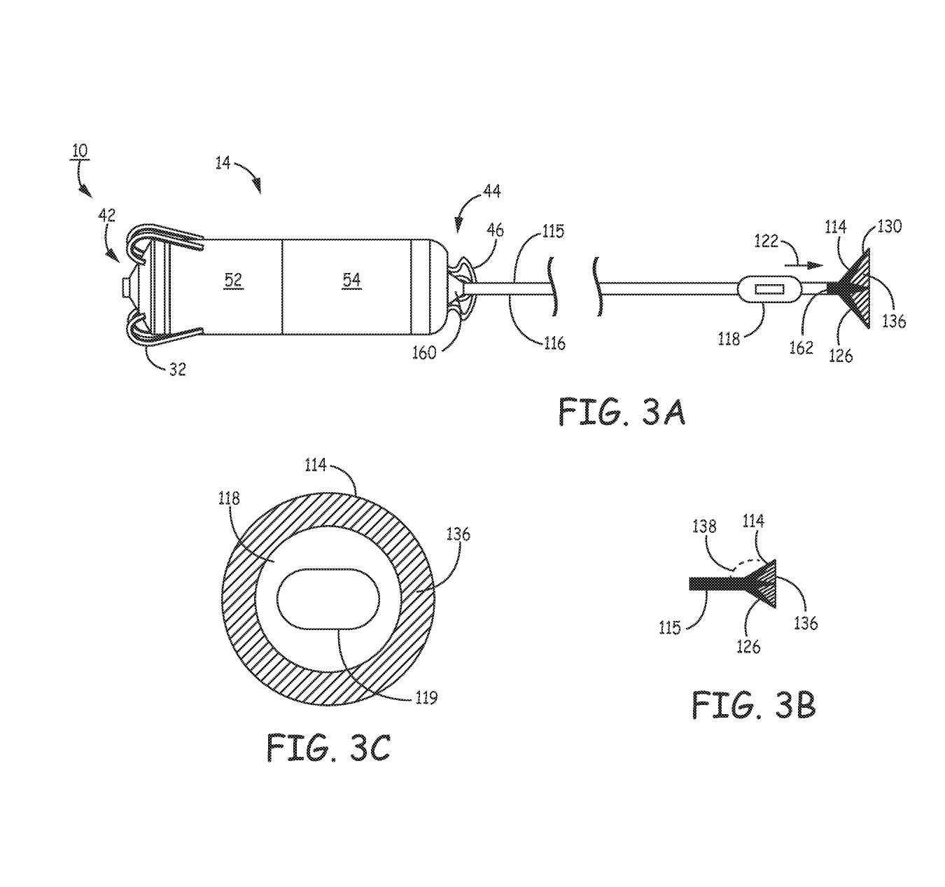

FIG. 3A is a conceptual diagram of IMD 10 including a sensor extension 116 carrying a pressure sensor 118 and floatation member 114 according to another example. Sensor extension 116 includes a sensor extension body 115 extending from a distal end 160 coupled to housing proximal end 44 to an extension proximal end 162 terminated by floatation member 114. Pressure sensor 118 is shown carried by extension body 115 distal to the proximally terminating floatation member 114. While not shown in FIG. 3A, sensor extension 116 may include one or more sensing electrodes carried along extension body 115 and/or IMD housing 14.

In this example, floatation member 114 is a tent- or umbrella-like structure. Floatation member 114 is pushed in a proximal direction away from housing 14 as indicated arrow 122 by the pressure of flowing blood acting against the distal surface area 126 of floatation member 114. Floatation member 114 has a distal surface area 126 against which the pressure of circulating blood acts upon to move extension proximal end 162 into the PA and maintains a substantially extended position of sensor extension 116 such that pressure sensor 118 is held within the PA due to pressure of circulating blood acting at least against distal surface area 126.

Floatation member 118 may be formed as a single molded piece that is permanently coupled to sensor extension proximal end 162. In some examples, floatation member 114 may include multiple struts 130 extending radially from a central attachment point to extension proximal end 162. A membrane 132 may extend between the struts 130 to define a continuous distal surface 126 against which flowing blood acts to push floatation member 114 away from housing 14 when housing 14 is anchored at a fixation site.

The distal surface 126 of flotation member 114 is shown in FIG. 3A to extend in a slightly proximal direction, i.e., at an obtuse angle from sensor extension body 115. In other examples, distal surface 126 may extend at other angles relative to extension body 115, including acute angles or a right angle. When distal surface 126 extends at an acute angle relative to sensor extension body 115, the orientation of the tent- or umbrella-like structure of floatation member 114 is inverted compared to the orientation shown in FIG. 3A.

Floatation member 114 is shown to be radially symmetric about sensor extension body 115 but may be asymmetric in other examples. An asymmetric floatation member 118 may cause extension proximal end 162 to be preferentially pushed in a direction toward the inner wall of the PA (or other blood vessel or heart chamber wall) rather than toward the center when blood flows against distal surface 126. When pushed toward the wall of the PA, pressure sensor 118 may be held in a more stable position as blood flows past floatation member 114.

The proximal face 136 of floatation member 114 is concave in some examples. While floatation member 114 is described as being a tent-like or umbrella-like structure such that proximal surface 136 is a concave surface in the orientation shown (or distal surface 126 is a concave surface in an inverted orientation), a solid cone shaped or pyramidal shape is contemplated that would have a substantially flat proximal surface 136 (or flat distal surface 126 when inverted relative to the orientation shown).

Floatation member 114 may be flexible or elastically deformable such that floatation member 114 may be compressed inward when held within a delivery tool. For example, floatation member 114 may be a self-expanding member that is held in a compressed position within a delivery tool, for example as generally shown in FIG. 3B, and expands to a normally-expanded position, as shown in FIG. 3A, when released from the delivery tool.

In other examples, the position of floatation member 114 shown in FIG. 3B may represent the normally expanded position of the floatation member 114 rather than a compressed position. The angle 138 between distal face 126 and extension body 115 may be relatively more obtuse than the angle shown in FIG. 3A when floatation member 114 is in a normal position. A relatively small surface area 126 normal to the high velocity blood flow along the RVOT (or other cardiovascular location) may be sufficient to push floatation member 114 away from housing 14 to a desired pressure monitoring site.

FIG. 3C is an end view of floatation member 114 according to another example. In this example, floatation member 114 and pressure sensor 118 are integrated such that the pressure sensor diaphragm 119 is exposed along the distal face 136 of floatation member 114. Floatation member 114 may be coupled to the extension proximal end 162 (shown in FIG. 3A). Pressure sensor 118 may be embedded or encased within floatation member 114 such that the pressure-sensitive diaphragm 119 remains exposed to circulating blood. In other examples, diaphragm 119 may be exposed along a distal face 126 of floatation member 114.

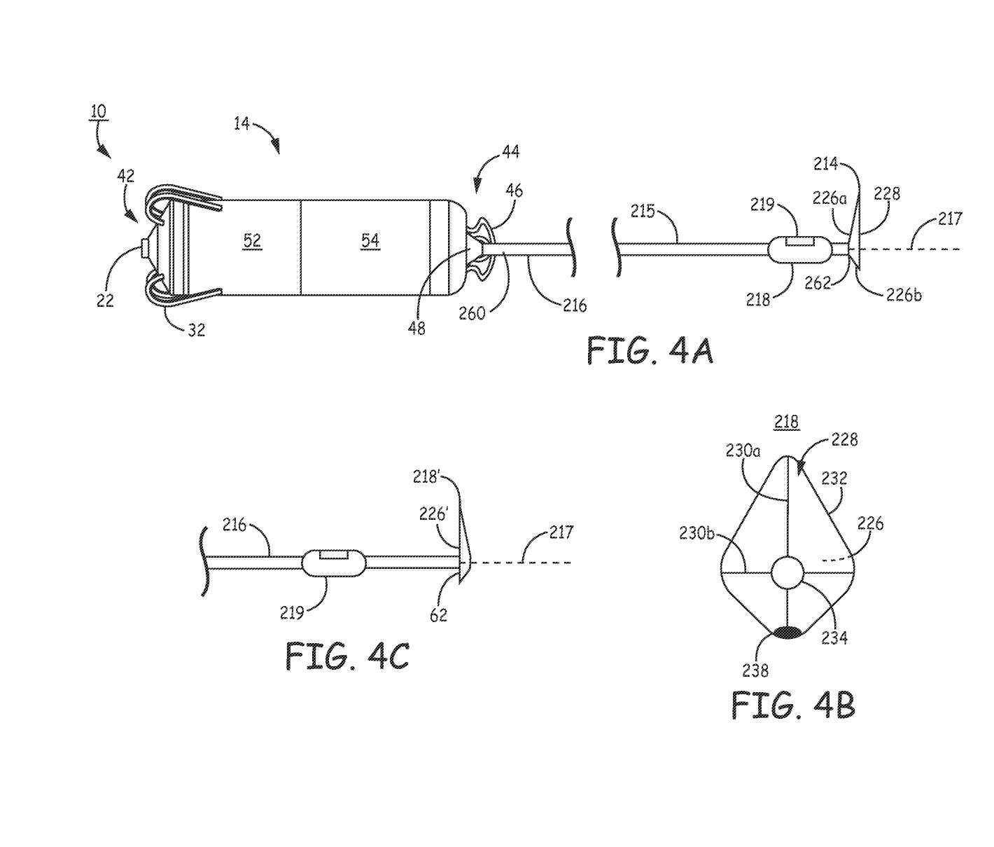

FIG. 4A is a conceptual diagram of IMD 10 according to another example in which a proximal sensor extension 216 terminates with a sail or kite-shaped floatation member 214. FIG. 4B is a proximal end view of floatation member 214. Floatation member 214 is asymmetric relative to a central support 234 that is coaxial with the central axis 217 of sensor extension body 215. Floatation member 214 may include perpendicular struts 230a and 230b, collectively 230, extending radially outward from center support 234. A membrane 232 extending between struts 230a and 230b defines a distal surface area 226 against which flowing blood applies a pressure that urges floatation member 214 away from housing 14, thereby extending pressure sensor 218 away from housing 14, e.g., into the PA.

Distal surface area 226 is asymmetrical including a larger surface area 226a (FIG. 4A) extending in one direction from center support 234 and a relatively smaller surface area 226b extending in an opposite direction from center support 234. A larger force will be applied by flowing blood against the larger surface area 226a resulting in a non-uniform pushing force relative to the central axis 217 of sensor extension body 215. When released into flowing blood, floatation member 214 may drift toward inner wall of a blood vessel or heart chamber, e.g., the PA inner wall, when the pressure of flowing blood acts to produce a larger total force on the larger distal surface area 226a.

Pressure sensor 218 may be carried along sensor extension body 215 and may be oriented such that the pressure-sensitive diaphragm 219 is exposed in a direction aligned with the larger distal surface area 226a. This orientation allows pressure sensor 218 to face away from an inner wall when floatation member 214 is urged toward the inner wall of a blood vessel, thereby maintaining optimal exposure to the pressure of circulating blood.

As shown in FIG. 4B, floatation member 214 may include a weighting member 238 positioned along the smaller surface area 226b, e.g., along an outer edge of membrane 232 or strut 230, to create unequal weighting of floatation member 214 with respect to central axis 217. Weighting member 238 may be a cylinder, bar, sphere, strip or other object that increases the weight of floatation member 214 along the minor or smaller surface area 226b relative to the weight of floatation member 218 along the major or larger surface area 226a. Floatation member 218 is preferentially driven toward a blood vessel or heart chamber inner wall due to the asymmetric weighting and sizes of surface areas 226a and 226b. Weighting member 238 may be a biocompatible metal, e.g., stainless steel or titanium alloys, which may be adhesively bonded to membrane 232 or overmolded during the formation of membrane 232. Weighting member 238 may be provided to offset the mass of pressure sensor 218 to provide a preferred directionality and behavior of sensor extension 216 when deployed in flowing blood.

A symmetrically weighted floatation member having a symmetric geometry may oscillate with the cardiac cycle when it remains in the central fluid path of the PA (or other blood vessel). The blood flow dynamics may drive an asymmetrically shaped and/or weighted flotation member 214 toward the vessel wall, e.g., toward the inferior PA wall near the left atrial roof. The asymmetric geometric shape and/or asymmetrical weighting of floatation member 214 caused by the addition of weighting member 238 may be designed to force the floatation member 214 toward the PA inner wall directing pressure sensor diaphragm 219 centrally within the vessel lumen. The blood velocity profile across a cross-section of a blood vessel may typically have the highest flow rate near the center of the vessel lumen. Exposure to higher velocity blood flow may reduce blood clotting and tissue encapsulation over diaphragm 218.

Floatation member 214 may be at least neutrally buoyant in blood even with the addition of weighting member 238. In other examples, weighting member 238 may be slightly negatively buoyant in blood such that the relatively more buoyant major surface area 226a floats “upward” as the weighted minor surface area 226b floats “downward” in the blood flow stream, causing the floatation member 214 to drift preferentially toward the PA (or other blood vessel) inner wall.

Additionally, the connection of floatation member 214 to extension body 215 at support member 234 being off-center relative to the floatation member geometry contributes to driving the floatation member 214 toward the PA inner wall. Alternatively, the floatation member 214 may be radially symmetric in geometry, such as floatation member 114 of FIG. 4A, and a weighting member 238 may be added along an outer edge of the radially symmetric floatation member to provide asymmetric weighting of the floatation member. A symmetric floatation member, such as floatation member 114, having asymmetric weighting may be coupled to extension body 215 coaxially or non-coaxially with central axis 217 of extension body 215.

In various examples, distal surface area 226 may be defined by a membrane 232 that is generally circular or polygonal in shape and supported by one or more struts and in some cases molded as a continuous, self-supporting structure without requiring supporting struts 230. While shown in an asymmetrical position with respect to a central axis 217 of sensor extension body 215, membrane 232 may have a geometry configured to be symmetrical relative to center support 234 and the central axis 217 of sensor extension body 215 in other examples.

Distal surface area 226 is shown as a generally convex surface, angled in a slightly proximal direction such that blood flowing against distal surface area 226 flows along and past floatation member 214. FIG. 4C is a partial view of sensor extension 216 according to another example in which sensor extension body 215 is terminated by floatation member 218′ having an orientation that is inverted proximally to distally compared to floatation member 217 such that distal surface area 226′ is a generally concave surface. Flowing blood is received within a concavity defined by distal surface area 226′. The asymmetrical configuration of floatation member 218′ may tip floatation member 218 toward an inner vessel wall causing sensor extension 216 to bend or curve toward the inner vessel wall, thereby urging pressure sensor diaphragm 219 toward centrally flowing blood.

Any of the floatation members described herein may include a bioabsorbable material so that the floatation member is wholly or partially absorbed into the blood stream, leaving the pressure sensor carried by the sensor extension positioned along a desired blood vessel. The partially or wholly bioabsorbable floatation member aids in floating the sensor extension 16 downstream and away from housing 14 upon deployment of IMD 10. Once pressure sensor 18 is positioned at a targeted pressure monitoring site, the floatation member may be partially or wholly absorbed over time. The use of an optional floatation member and its properties (shape, symmetry, weighting, bioabsorbability, etc.) in combination pressure sensor 18 may be based upon the particular flow dynamics expected to be encountered at a deployment site and at the targeted pressure monitoring site.

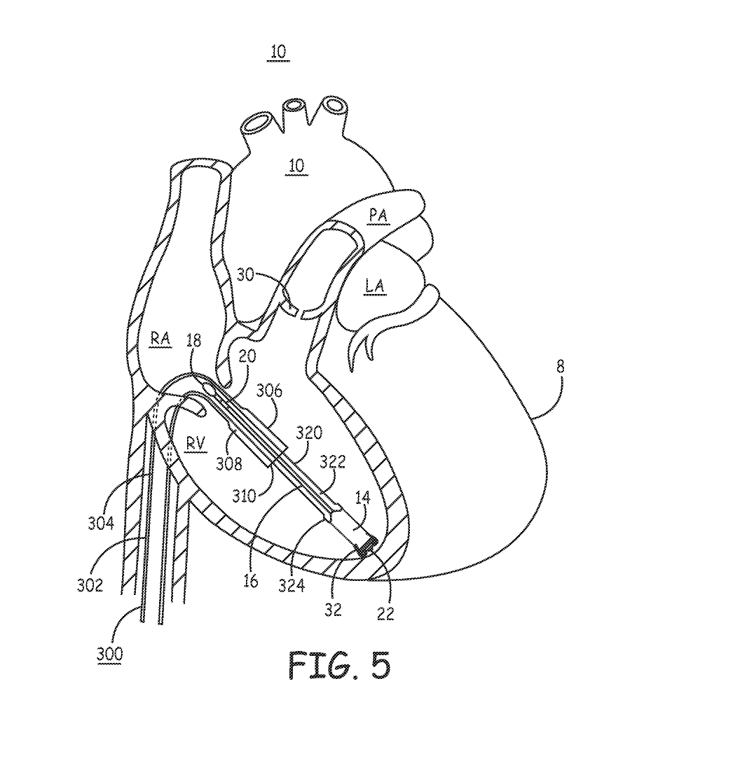

FIG. 5 is a conceptual diagram of a delivery tool 300 and IMD 10 as it is deployed into the RV. Delivery tool 300 may include an outer catheter 302 defining an outer lumen 304 through which an inner catheter 320 extends. Inner catheter 320 defines an inner lumen 322 in which proximal sensor extension 16 extends. Prior to deployment of IMD 10, housing 14 may be retained within a delivery tool capsule 306 that defines a cavity 308 for retaining housing 14 during advancement of outer catheter 302 into the RV. Inner catheter 320 may be fully withdrawn into outer lumen 304 such that housing 14 is retained within capsule 308.

With housing 14 retained within capsule 308, delivery tool 300 may be advanced transvenously into the RA, e.g., via the inferior vena cava in the example shown, and advanced further into the RV. Once within the RV or in proximity of a target fixation site, inner catheter 320 may be advanced distally out a distal opening 310 of delivery tool 300 and/or outer catheter 302 may be withdrawn proximally relative to inner catheter 320. Inner catheter 320 may include a distal cone or cup 324 configured to interface with the proximal end 44 of housing 14 for advancing housing distal end 42 out distal opening 310 and against a target fixation site. Fixation member 32, which may be held in an extended position as shown in FIG. 2B, is deployed as housing 14 is released from capsule 306. In some examples, the distal opening 310 is placed adjacent a target fixation site. Fixation member 32 may be held in an extended position within capsule 306. Inner catheter 320 is advanced as outer catheter 302 is retracted such that distal tips of tines 41 included in fixation member 32 pierce into the ventricular endocardial tissue then curve proximally to regain a relaxed position as shown, capturing tissue to actively fix housing 14 at the target fixation site.

After housing 14 is fixed at the fixation site, inner catheter 320 may be used to manipulate or steer sensor extension 16 to a desired location for release into circulating blood to deploy pressure sensor 18 near a pressure monitoring site or upstream from a targeted pressure monitoring site. Inner catheter 320 may be a steerable catheter in some examples, including a pull wire or other mechanism to steer the distal end of inner catheter 320 to a desired location for releasing sensor extension 16.

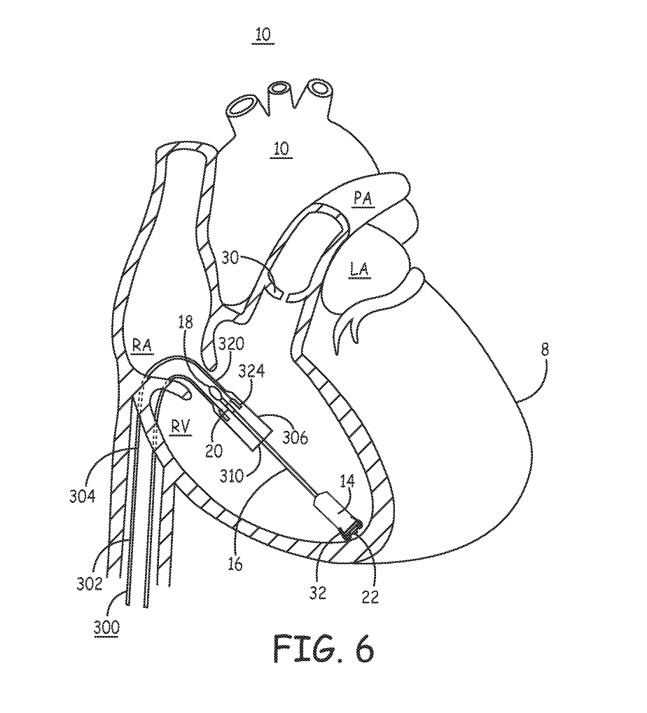

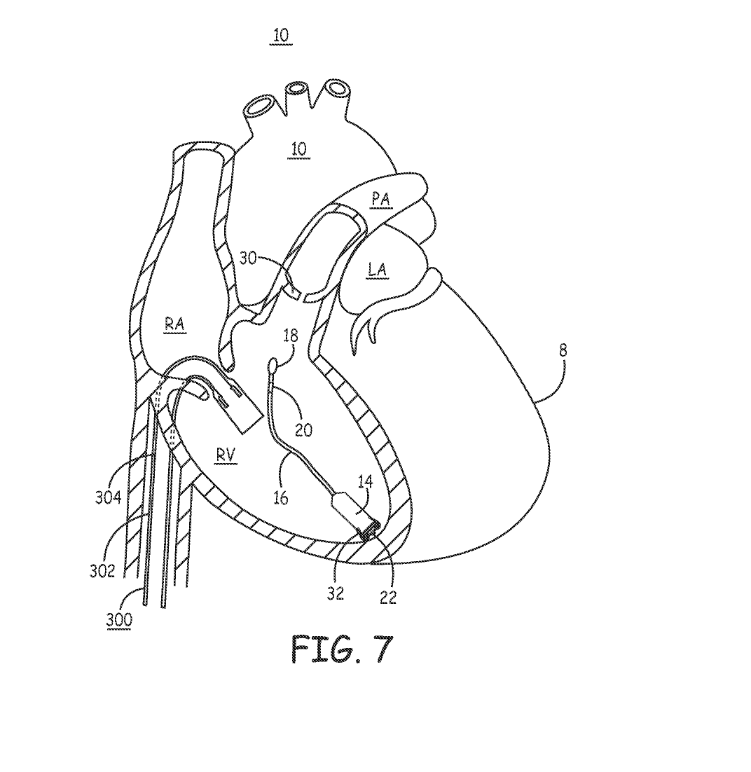

FIG. 6 depicts IMD 10 after inner catheter 320 has been retracted back into outer catheter 302. Distal cup 324 of inner catheter 320 is retracted back into capsule 306 of outer catheter 302. In this instance, a portion of sensor extension 16 extends within delivery tool 300 such that delivery tool 300 may be advanced or manipulated as needed to position sensor extension 16 at a desired deployment location, e.g., within the RV along the RVOT. Delivery tool 300 is retracted to fully release IMD 10 from delivery tool distal opening 310 such that sensor extension 16 is released from delivery tool 300 as shown in FIG. 7. Pressure sensor 18 will be released into the RV blood pool and enter the RVOT as circulating blood in the RV acts on pressure sensor 18 (and a floatation member if present).

Pressure sensor 18 caught in the circulating blood in the RV will enter the RVOT and be pushed into the PA by the pressure of blood acting on the pressure sensor 18 (and floatation member if present) thereby extending sensor extension 16 toward the PA, possibly crossing the pulmonary valve 30, to a position pressure sensor 18 in the PA as illustrated in FIG. 1. The fixation of housing 14 by fixation member 32 counteracts the force of circulating blood acting on pressure sensor 18 to maintain housing 14 at the target fixation site while pressure sensor 18 is maintained at a target pressure monitoring site in the PA without drifting further downstream.

In the examples shown, the overall length of sensor extension 16 relative to heart 8 and housing 14 may not be drawn to scale. It is recognized that the length of sensor extension 16 is provided as needed to position pressure sensor 18 at a targeted pressure monitoring location spaced apart for a targeted fixation site. For example, extension 16 is relatively shorter for positioning sensor 18 within the RVOT or relatively longer for crossing pulmonary valve 30 for positioning sensor 18 within the PA.

It is further recognized that in other examples, the delivery tool 300 may include a third, innermost catheter extending within inner catheter 320. Sensor extension 16 may extend within the third innermost catheter which may be used to deliver sensor extension 16 to a desired pressure monitoring site spaced apart from the implant site of housing 14.

Pressure sensor 18 is maintained at the pressure monitoring site, in the PA in this example, by a single fixation member 32 on the housing distal end 42, which may be anchored in a different heart chamber or blood vessel location than the pressure monitoring site. Sensor extension 16 may be provided without an active or passive fixation member such that the action of flowing blood maintains pressure sensor 18 at a position spaced apart from the fixation site of housing 14, without requiring actively fixing IMD 14 at the pressure monitoring site.

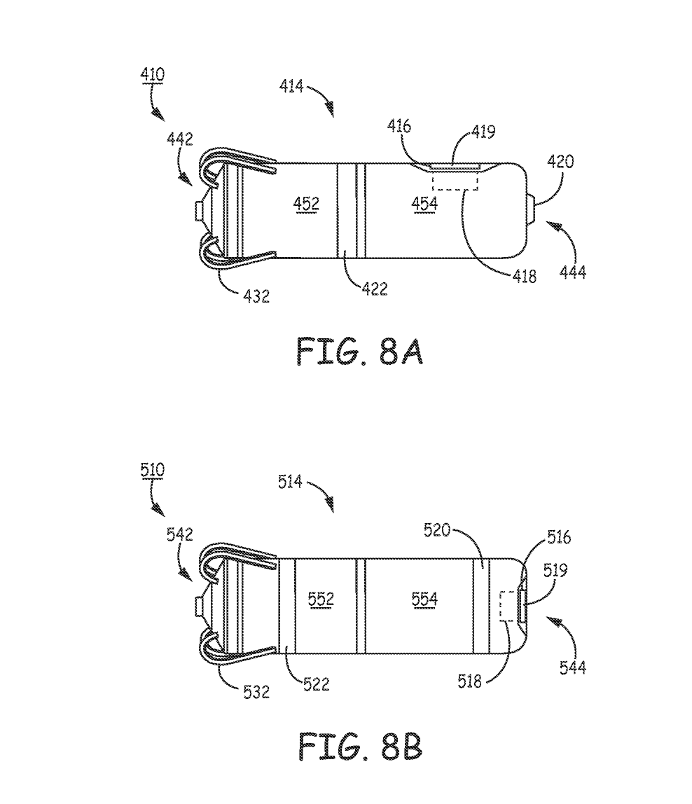

FIGS. 8A and 8B are conceptual diagrams of alternative examples of an IMD having a pressure sensor incorporated within the housing. In FIG. 8A, control electronics 454 includes a pressure sensor 418. Housing 414 includes an opening 416 to expose pressure-sensitive diaphragm 419 that transfers pressure exerted on diaphragm 419 by circulating blood to a gap capacitor or other pressure transducing electronics included in pressure sensor 418. Housing 414 extends between a housing proximal end 444 and housing distal end 442 and includes a battery subassembly 452 defining a distal portion of the housing 414 in this example.

IMD 410 may include a proximal tip electrode 420 and a ring electrode 422. Tip electrode 420 may be coupled to circuitry within control electronics subassembly 454 via an electrical feedthrough crossing housing 414. Ring electrode 422 may be coupled to housing 414 to serve as a return anode electrode when paired with tip electrode 420 for sensing cardiac electrical signals and delivering electrical stimulation pulses when IMD 410 includes therapy delivery capabilities.

Fixation member 432 is located at housing distal end 442 for anchoring housing 414 at a fixation site. The fixation site in this example is near the pressure monitoring site such that upon fixation of housing 414, pressure sensor 418 is positioned at the pressure monitoring site, e.g., within a heart chamber, along the RVOT, or within a blood vessel. When pressure sensor 418 (or any of the pressure sensors disclosed herein) is positioned within the right ventricle or along RVOT, the right ventricular pressure signal may be used to estimate pulmonary artery pressure, e.g., as generally disclosed in U.S. Pat. No. 6,865,419 (Mulligan, et al.), incorporated herein by reference in its entirety. Electrodes 420 and 422 may be used to sense a cardiac electrical event, e.g., an R-wave so that pressure may be determined at particular time point(s) in the cardiac cycle relative to the R-wave.

In FIG. 8A, pressure sensor diaphragm 419 is shown exposed along a circumferential side of housing 514. In FIG. 8B, IMD 510 includes a pressure sensor 518 having a pressure-sensitive diaphragm 519 that is exposed through a proximal opening 516 along the housing proximal end 544 for monitoring pressure. Pressure sensor 518 is incorporated in control electronics assembly 554. Fixation of IMD housing 514 by fixation member 532 anchors IMD 510 at a desired pressure monitoring site.

IMD 510 may optionally include a pair of ring electrodes along housing 514. For example, a cathode ring electrode may be located along a proximal portion of control electronics subassembly 554 and an anode ring electrode may be located distally along battery subassembly 552 for monitoring cardiac electrical signals and delivering cardiac electrical stimulation pulses when IMD 510 includes therapy delivery capabilities.

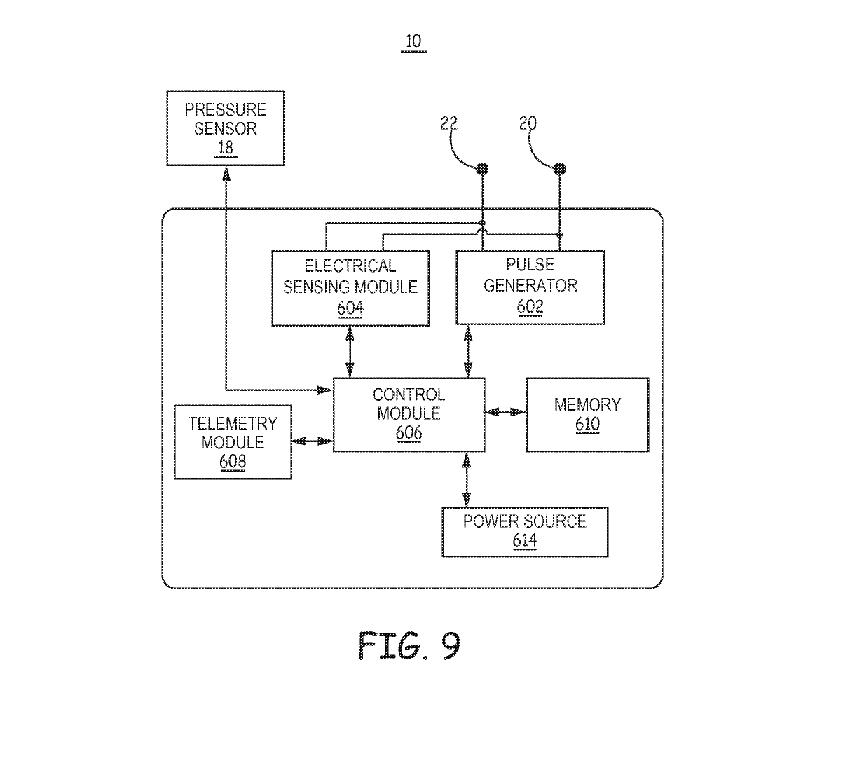

FIG. 9 is a block diagram of IMD 10 of FIG. 1 according to one example and is representative of the functionality of any of the illustrative examples of IMDs shown and described herein. The block diagram and functionality attributed to IMD 10 may be associated with any of the example IMD configurations described above. IMD 10 may include a pulse generator 602, an electrical sensing module 604, a control module 606, memory 610, telemetry module 608 and a power source 614. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

The functions attributed to IMD 10 herein may be embodied as one or more processors, controllers, hardware, firmware, software, or any combination thereof. Depiction of different features as specific circuitry or modules is intended to highlight different functional aspects and does not necessarily imply that such functions must be realized by separate hardware or software components or by any particular architecture. Rather, functionality associated with one or more modules, processors, or circuits may be performed by separate hardware or software components, or integrated within common hardware or software components. For example, pressure monitoring operations performed by IMD 10 may be implemented in control module 606 executing instructions stored in associated memory 610 and may rely on timing-related input from electrical sensing module 604.

The functional operation of IMD 10 as disclosed herein should not be construed as reflective of a specific form of software or hardware necessary to practice the methods described. It is believed that the particular form of software, hardware and/or firmware will be determined primarily by the particular system architecture employed in the IMD 10 and by the particular sensing and therapy delivery methodologies employed by the IMD 10. Providing software, hardware, and/or firmware to accomplish the described functionality in the context of any modern IMD system, given the disclosure herein, is within the abilities of one of skill in the art.

Pressure sensor 18 is shown coupled to control module 606 (via sensor extension 16 and any necessary electrical feedthroughs as described in conjunction with FIG. 2A or via a hybrid circuit when pressure sensor 18 is incorporated within IMD housing 14, as shown in FIG. 8A and 8B). Control module 606 receives an electrical signal from pressure sensor 18 correlated to the pressure of circulating blood exerted on sensor 18 and may store pressure signal episodes in memory 610 and/or determine pressure parameters, such as systolic pressure, diastolic pressure, average pressure, dP/dt, or other desired pressure monitoring parameters. In some examples, IMD 10 is solely a pressure monitoring device. In other examples, IMD 10 may include cardiac electrical signal monitoring and/or therapy delivery capabilities.

Pulse generator 602, if included for therapy delivery purposes, is configured to generate electrical stimulation pulses that may be delivered to heart tissue via electrodes 20 and 22. Pulse generator 602 may include one or more capacitors and a charging circuit to charge the capacitor(s) to a programmed pacing pulse voltage. At appropriate times, as controlled by a pace timing and control module included in control module 606, the capacitor is coupled to electrodes 20 and 22 to discharge the capacitor voltage and thereby deliver the pacing pulse. Pacing circuitry generally disclosed in the above-incorporated U.S. Pat. No. 5,507,782 (Kieval, et al.) and in commonly assigned U.S. Pat. No. 8,532,785 (Crutchfield, et al.), also incorporated herein by reference in its entirety, may be implemented in IMD 10 for charging a pacing capacitor to a predetermined pacing pulse amplitude under the control of control module 606 and delivering a pacing pulse.

Electrical sensing module 604 may be configured to receive cardiac electrical signals developed across electrodes 20 and 22. A cardiac event may be sensed by sensing module 604 when the cardiac electrical signal crosses a sensing threshold of a cardiac event detector included in sensing module 604, such as a sense amplifier. The sensing threshold may be an auto-adjusting sensing threshold that may be initially set based on the amplitude of a sensed event and decays at a predetermined decay rate thereafter. In response to a sensing threshold crossing, electrical sensing module 604 passes a sensed event signal to control module 606.

Memory 610 may include computer-readable instructions that, when executed by control module 606 cause control module 606 to perform pressure monitoring algorithms. The computer-readable instructions may be encoded within memory 610. Memory 610 may include any non-transitory, computer-readable storage media including any volatile, non-volatile, magnetic, optical, or electrical media, such as a random access memory (RAM), read-only memory (ROM), non-volatile RAM (NVRAM), electrically-erasable programmable ROM (EEPROM), flash memory, or other digital media with the sole exception being a transitory propagating signal. Memory 610 stores timing intervals, counters, or other data used by control module 606 to monitor one or more pressure parameters or record pressure signals according to an implemented pressure monitoring algorithm, e.g., according to an algorithm for monitoring PAP or for estimating PAP from a RV pressure signal.

Power source 614 provides power to each of the other modules and components of IMD 10 as required. Power source 614 may include one or more energy storage devices, such as one or more rechargeable or non-rechargeable batteries. The connections between power source 614 and other modules and components are not shown in FIG. 9 for the sake of clarity.

Telemetry module 608 includes a transceiver and associated antenna for transferring and receiving data via a radio frequency (RF) communication link. Telemetry module 308 may be capable of bi-directional communication with an external device 36 as described in conjunction with FIG. 1. Additionally, IMD 10 may communicate via telemetry module 308 with another IMD such as another therapy delivery or monitoring device implanted in the patient.

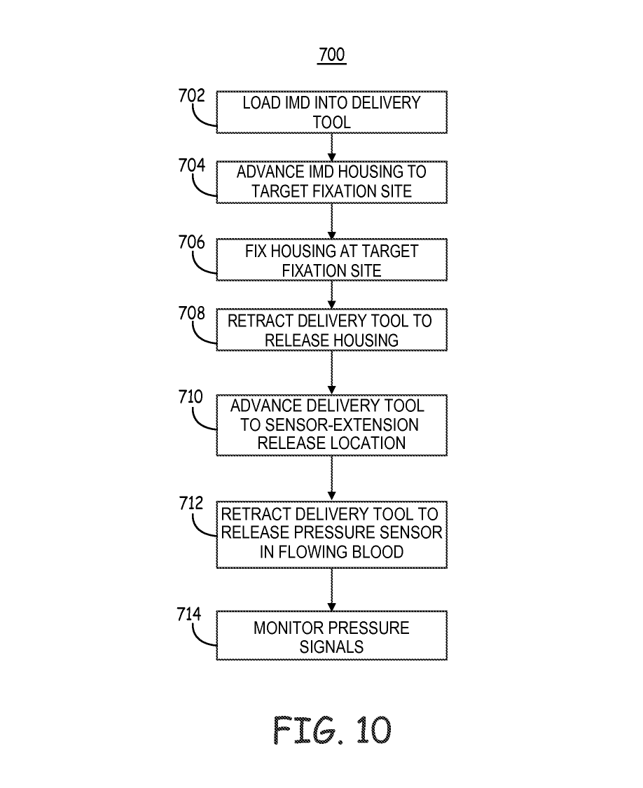

FIG. 10 is a flow chart 700 of a method for deploying IMD 10 for monitoring a pressure signal. At block 702, IMD 10 is loaded into a delivery tool including a lumen for receiving proximal sensor extension 16 and a capsule for receiving and retaining the IMD housing 14. The delivery tool is advanced to a target fixation site at block 704 that is the implant site of the IMD housing 14. At block 706, the IMD housing 14 is fixed at the target site, e.g., by advancing IMD housing 14 out a distal opening of the delivery tool to cause the distal fixation member 32 to engage tissue at the target site as described above in conjunction with FIG. 5.

With the IMD housing 14 fixed at the target fixation site, the delivery tool may be retracted at block 708 to fully release IMD housing 14 from the delivery tool. The proximal sensor extension 16 may remain at least partially within the delivery tool lumen such that the delivery tool may be used to position the sensor extension 16 at a desired release site in flowing blood (block 710), e.g., within the RV or along the RVOT as described above in conjunction with FIG. 6. The delivery tool may be fully retracted to release the sensor extension floatation member at block 712. At this point, the IMD 10 may be fully released from the delivery tool. The pressure sensor 18 released into the flowing blood is subjected to pressure of circulating blood that moves the pressure sensor 18 away from the housing 14 such that the sensor extension 16 extends away from housing 14 thereby positioning pressure sensor 18 at a desired pressure monitoring site, using only a single housing-based fixation member which is positioned along the housing distal end in some examples, without requiring a fixation member along the pressure sensor 18 or the sensor extension 16.

At block 714, the extension-based pressure sensor 18 that is “floated” away from housing 14 is used for monitoring pressure signals. The control module of IMD 10 receives a pressure signal from the pressure sensor 18 and may store pressure signals and/or determine pressure monitoring parameters from the received signal.

Thus, various examples of an implantable medical device including a pressure sensor have been described. It is recognized that various modifications may be made to the described embodiments without departing from the scope of the following claims.

1. An implantable medical device, comprising:
a housing having a proximal end and a distal end;
a control module enclosed by the housing;
a pressure sensor electrically coupled to the control module; and
a fixation member coupled to the housing distal end for anchoring the housing distal end at a fixation site within a cardiovascular system of a patient,
the pressure sensor spaced apart proximally from the fixation member.

2. The device of claim 1, further comprising an electrical extension having a distal end, a proximal end and an elongate body extending from the extension distal end to the proximal end,
the extension distal end coupled to from the housing proximal end,
the proximal end configured to float in flowing blood downstream from the housing when the housing distal end is anchored at the fixation site by the fixation member,
the pressure sensor carried by the electrical extension.

3. The device of claim 2, wherein the pressure sensor is carried at the proximal end of the electrical extension.

4. The device of claim 2, wherein the extension is configured to extend along a right ventricular outflow tract when the housing distal end is anchored in a right ventricle of the patient to position the pressure sensor within a pulmonary artery;
wherein the control module is configured to determine a pulmonary artery pressure from the signal.

5. The device of claim 2, wherein the extension includes a floatation member that is at least neutrally buoyant in blood.

6. The device of claim 5, wherein the pressure sensor is incorporated in the floatation member.

7. The device of claim 5, wherein the floatation member is at least partially bioabsorbable.

8. The device of claim 5, wherein the floatation member is configured to preferentially float toward an inner wall of the cardiovascular system by having at least one of a weight and a surface area that is asymmetric relative to a central axis of the electrical extension,
the pressure sensor comprising a pressure-sensitive diaphragm along the elongate body that is directed away from the inner wall when the floatation member preferentially floats toward the inner wall.

9. The device of claim 2, wherein the elongate body comprises a first stiffness adjacent the extension distal end and a second stiffness adjacent the extension proximal end, the first stiffness greater than the second stiffness.

10. The device of claim 1, wherein the pressure sensor comprises a pressure-sensitive diaphragm and the housing comprises a lateral sidewall extending between the housing proximal end and the housing distal end and an opening configured to expose the pressure-sensitive diaphragm along one of the lateral sidewall and the housing proximal end.

11. The device of claim 1, further comprising:
a pair of electrodes; and
a sensing module enclosed by the housing and configured to sense cardiac electrical signals via the pair of electrodes.

12. A system, comprising:
an implantable medical device comprising:
a housing having a proximal end and a distal end;
a control module enclosed by the housing;
a pressure sensor electrically coupled to the control module,
a fixation member coupled to the housing distal end for anchoring the housing distal end at a fixation site within a cardiovascular system of a patient,
the pressure sensor spaced apart proximally from the fixation member;
and
a delivery tool comprising a cavity for receiving the housing and for advancing the housing distal end to the fixation site.

13. The system of claim 12, further comprising:
an electrical extension having a distal end, a proximal end and an elongate body extending from the extension distal end to the proximal end,
the extension distal end coupled to the housing proximal end,
the extension proximal end configured to float in flowing blood downstream from the housing when the housing distal end is anchored at the fixation site by the fixation member,
the pressure sensor carried by the electrical extension,
the delivery tool comprising a lumen for receiving the electrical extension extending from the housing proximal end and configured to release the proximal end into flowing blood to allow the extension to extend away from the housing along a direction of the flowing blood to position the pressure sensor at a target pressure monitoring site.

14. The system of claim 13, wherein the pressure sensor is carried at the proximal end of the electrical extension.

15. The system of claim 13, wherein the electrical extension is configured to extend along a right ventricular outflow tract when the housing distal end is anchored in a right ventricle of the patient to position the pressure sensor within a pulmonary artery;
wherein the control module is configured to determine a pulmonary artery pressure parameter from the signal.

16. The system of claim 13, wherein the extension includes a floatation member that is at least neutrally buoyant in blood.

17. The system of claim 16, wherein the pressure sensor is incorporated in the floatation member.

18. The system of claim 16, wherein the floatation member is at least partially bioabsorbable.

19. The system of claim 16, wherein the floatation member is configured to preferentially float toward an inner wall of the cardiovascular system by having at least one of a weight and a surface area that is asymmetric relative to a central axis of the electrical extension,
the pressure sensor comprising a pressure-sensitive diaphragm along the elongate body that is directed away from the inner wall when the floatation member preferentially floats toward the inner wall.

20. The system of claim 13, wherein the elongate body comprises a first stiffness adjacent the extension distal end and a second stiffness adjacent the extension proximal end, the first stiffness greater than the second stiffness.

21. The system of claim 12, wherein the pressure sensor comprises a pressure-sensitive diaphragm and the housing comprises a lateral sidewall extending between the housing proximal end and the housing distal end and an opening configured to expose the pressure-sensitive diaphragm along one of the lateral sidewall and the housing proximal end.

22. The system of claim 12, further comprising:
a pair of electrodes;
a sensing module enclosed by the housing and configured to sense cardiac electrical signals via the pair of electrodes.

23. An implantable medical device, comprising:
a housing having a proximal end and a distal end;
a control module enclosed by the housing;
an electrical extension having a proximal end, a distal end coupled to the housing proximal end, and an elongate body extending from the distal end to the proximal end;
a pressure sensor carried by the electrical extension elongate body and spaced apart from the housing, the pressure sensor electrically coupled to the control module via the electrical extension; and
a fixation member for anchoring the implantable medical device at a fixation site within a cardiovascular system of a patient, the fixation member coupled to the housing distal end.
